(19) United States
(12) Patent Application Publication
(10) Pub. No.: US 2026/0128614 A1
(43) Pub. Date: May 7, 2026

(54) PARASITIC ELECTROMAGNETIC FIELD SWITCH FOR SEALED BARRIER APPLICATIONS

(71) Applicant: Cilag GmbH International, Zug (CH)

(72) Inventors: Frederick E. Shelton, IV, Hillsboro, OH (US); Shane R. Adams, Lebanon, OH (US)

(21) Appl. No.: 18/939,504

(22) Filed: Nov. 6, 2024

(51) Int. Cl. H01H 9/04; H01H 36/00

(52) U.S. Cl. CPC H02J 50/10; H01F 7/204; A61B 2017/00221; A61B 17/29

(57) ABSTRACT

An electronic medical device includes a disposable housing and a primary circuit that interacts with external secondary circuits through magnetic coupling. The primary circuit, housed in a sealed, removable unit, includes an inductor and capacitor forming a resonant circuit monitored by a connected circuit that detects electrical characteristics. Positioned near the primary circuit, the secondary circuit contains an electrical actuator with its own resonant circuit. The device operates via mutual inductance between the primary and secondary circuits, with the monitor detecting changes in response based on the actuator's state. A frequency-swept alternating current (AC) signal is used to measure these characteristics, allowing the detection of actuator states. The system can be expanded with additional secondary circuits, enabling further magnetic coupling and monitoring. The device's design facilitates activation and monitoring of multiple actuators through resonant frequency responses for precise control and feedback.

### BACKGROUND

Disposable electronic medical devices offer convenience but generate significant waste. To address this issue, it is desirable to separate the device into disposable and reusable components. To be viable, the reusable components must withstand sterilization or autoclaving.

For optimal design, the housing and handle of the medical device should be disposable, while the electronic components, which are costly to replace, should be reusable. To achieve this, the electronic components must be sealed within a housing that withstands sterilization or autoclaving. This approach reduces waste and enhances the device's cost-effectiveness.

However, a challenge arises because the electronic components need to receive input from a user operated electrical actuator located on an exterior wall of the housing to control the operation of motors and other electronic functions. Wired connections are impractical in this scenario, as the housing containing the electronic control module must be sealed to survive the sterilization and/or autoclaving process.

### SUMMARY

In a first embodiment, the present disclosure describes an electronic medical device including a disposable housing, a primary circuit, and a secondary circuit. The primary circuit is disposed in a sealed housing removably connectable to the disposable housing. The primary circuit including a primary inductor with inductance L₁ to generate a magnetic field in response to applied electrical energy. The secondary circuit is positioned on an exterior wall of the disposable housing and proximal to the primary circuit. The secondary circuit includes an electrical actuator that includes a secondary inductor with inductance L₂ arranged in proximity to the primary inductor to induce electrical energy in the secondary inductor via mutual inductance M when the primary inductor is energized. The electrical actuator is operable to control the electrical flow through the secondary inductor based on the induced electrical energy. Magnetic coupling between the primary inductor and the secondary inductor is defined by the mutual inductance M. The electrical actuator is configured to control energy transfer from the primary inductor to the secondary inductor to control the induced electrical energy in the secondary circuit.

In conjunction with one or more embodiments of the first embodiment, the primary circuit includes a direct current (DC) voltage source to energize the primary inductor.

In conjunction with one or more embodiments of the first embodiment, the primary circuit comprises an alternating current (AC) voltage source to energize the primary inductor.

In conjunction with one or more embodiments of the first embodiment, the electrical actuator comprises a switch connected in series or in parallel with the secondary inductor.

In conjunction with one or more embodiments of the first embodiment, the electrical actuator comprises a switch connected in series or in parallel with the secondary inductor and the electrical actuator includes an elastomer mechanically linked to the switch.

In conjunction with one or more embodiments of the first embodiment, the electrical actuator comprises a switch connected in series or in parallel with the secondary inductor, the electrical actuator includes an elastomer mechanically linked to the switch, and the electrical actuator is affixed to the exterior wall of the disposable housing with an adhesive.

In a second embodiment, the present disclosure describes an electronic medical device including a disposable housing, a primary circuit, and a secondary circuit. The primary circuit is disposed in a sealed housing removably connectable to the disposable housing. The primary circuit includes a primary inductor with inductance L₁ to generate a magnetic field in response to applied electrical energy. The primary circuit also includes a monitor circuit to detect changes in electrical characteristics of the primary inductor. The secondary circuit is positioned on an external wall of the disposable housing and proximal to the primary circuit. The secondary circuit includes an electrical actuator that includes a secondary inductor with inductance L₂ positioned in proximity to the primary inductor to induce mutual inductance M between the primary inductor and the secondary inductor. The electrical actuator is operable to control electrical flow through the secondary inductor based on induced voltage. The changes in the electrical characteristics of the primary inductor are based on an activation state of the electrical actuator.

In conjunction with one or more embodiments of the second embodiment, the monitor circuit detects the changes in electrical characteristics of the primary circuit, including changes in voltage, current, impedance, frequency, or phase, or combinations thereof, based on whether the electrical actuator is open or closed.

In conjunction with one or more embodiments of the second embodiment, the primary circuit includes a direct current (DC) source to energize the primary inductor or an alternating current (AC) source to energize the primary inductor.

In conjunction with one or more embodiments of the second embodiment, the electrical actuator includes a switch and a frequency tuning element.

In conjunction with one or more embodiments of the second embodiment, the electrical actuator includes a switch and a frequency tuning element and the switch is connected in series or in parallel with the frequency tuning element.

In a third embodiment, the present disclosure describes an electronic medical device including a disposable housing, a primary circuit, and a secondary circuit. The primary circuit is disposed in a sealed housing removably connectable to the disposable housing. The primary circuit includes a first inductor L₁ and a first capacitor C₁ to form a primary resonant circuit with a resonant frequency f₁. The primary circuit also includes a monitor circuit operatively connected to the primary resonant circuit to measure an electrical characteristic of the primary resonant circuit. The secondary circuit is positioned on an exterior wall of the disposable housing arranged in proximity to the primary circuit to enable magnetic coupling through mutual inductance M between the primary and secondary circuits. The secondary circuit includes an electrical actuator including a second inductor L₂ and a second capacitor C₂ to form a second resonant circuit with a resonant frequency f₂. The electrical actuator activates the secondary circuit. The primary circuit and the secondary circuit are coupled by mutual inductance M based on a state of the electrical actuator. The monitor circuit detects a response of the primary circuit based on the state of the electrical actuator.

In conjunction with one or more embodiments of the third embodiment, the electronic medical device includes a frequency-swept alternating current (AC) signal source to generate a signal with a frequency that varies over a predetermined range, including the resonant frequency f₁.

In conjunction with one or more embodiments of the third embodiment, the electronic medical device includes a frequency-swept alternating current (AC) signal source to generate a signal with a frequency that varies over a predetermined range, including the resonant frequency f₁ and the monitor circuit detects the electrical characteristics of the primary circuit over the frequency range of the signal.

In conjunction with one or more embodiments of the third embodiment, the electronic medical device includes a frequency-swept alternating current (AC) signal source to generate a signal with a frequency that varies over a predetermined range, including the resonant frequency f₁ and the primary resonant circuit exhibits a first response when the electrical actuator is open and a second response when it is closed, and the monitor circuit detects changes between the first and second responses.

In conjunction with one or more embodiments of the third embodiment, the electronic medical device includes a frequency-swept alternating current (AC) signal source to generate a signal with a frequency that varies over a predetermined range, including the resonant frequency f₁ and the primary resonant circuit exhibits a first response when the electrical actuator is open and a second response when it is closed, and the monitor circuit detects changes between the first and second responses and the monitor circuit detects a state of the electrical actuator based on the changes between the first and second responses during the frequency sweep.

In conjunction with one or more embodiments of the third embodiment, the electronic medical device includes an additional secondary circuit positioned on an exterior wall of the disposable housing arranged in proximity to the primary circuit to enable magnetic coupling through mutual inductance M between the primary and the additional secondary circuits. The additional secondary circuit includes a second electrical actuator including a third inductor L₃ and a third capacitor C₃ to form a third resonant circuit with a resonant frequency f₃. The second electrical actuator activates the additional secondary circuit. The primary circuit and the additional secondary circuit are coupled by mutual inductance M based on the state of the second electrical actuator. The monitor circuit detects a response of the primary circuit based on the state of the second electrical actuator.

In conjunction with one or more embodiments of the third embodiment, the electronic medical device includes an additional secondary circuit positioned on an exterior wall of the disposable housing arranged in proximity to the primary circuit to enable magnetic coupling through mutual inductance M between the primary and the additional secondary circuits. The additional secondary circuit includes a second electrical actuator including a third inductor L₃ and a third capacitor C₃ to form a third resonant circuit with a resonant frequency f₃. The second electrical actuator activates the additional secondary circuit. The primary circuit and the additional secondary circuit are coupled by mutual inductance M based on the state of the second electrical actuator. The monitor circuit detects a response of the primary circuit based on the state of the second electrical actuator. The primary resonant circuit exhibits a first response when the second electrical actuator is open and a second response when it is closed, and wherein the monitor circuit detects changes between the first and second responses.

In conjunction with one or more embodiments of the third embodiment, the electronic medical device includes an additional secondary circuit positioned on an exterior wall of the disposable housing arranged in proximity to the primary circuit to enable magnetic coupling through mutual inductance M between the primary and the additional secondary circuits. The additional secondary circuit includes a second electrical actuator including a third inductor L₃ and a third capacitor C₃ to form a third resonant circuit with a resonant frequency f₃. The second electrical actuator activates the additional secondary circuit. The primary circuit and the additional secondary circuit are coupled by mutual inductance M based on the state of the second electrical actuator. The monitor circuit detects a response of the primary circuit based on the state of the second electrical actuator. The primary resonant circuit exhibits a first response when the second electrical actuator is open and a second response when it is closed, and wherein the monitor circuit detects changes between the first and second responses. The monitor circuit detects a state of the second electrical actuator based on the changes between the first and second responses during the frequency sweep.

In conjunction with one or more embodiments of the third embodiment, the electronic medical device includes a second primary circuit and a second secondary circuit. The second primary circuit includes a third inductor L₃ and a third capacitor C₃ to form a third resonant circuit with a resonant frequency f₃. The second primary circuit also includes a second monitor circuit operatively connected to the third resonant circuit to measure an electrical characteristic of the third resonant circuit. The second secondary circuit is positioned on an exterior wall of the disposable housing arranged in proximity to the second primary circuit to enable magnetic coupling through mutual inductance M between the second primary and second secondary circuits. The second secondary circuit including a second electrical actuator including a fourth inductor L₄ and a fourth capacitor C₄ to form a fourth resonant circuit with a resonant frequency f₄. The second electrical actuator activates the second secondary circuit. The second primary circuit and the second secondary circuit are coupled by mutual inductance M based on the state of second electrical actuator. The second monitor circuit detects a response of the second primary circuit based on the state of the second electrical actuator.

### BRIEF DESCRIPTION OF THE DRAWINGS

In the description, for purposes of explanation and not limitation, specific details are set forth, such as particular aspects, procedures, techniques, etc. to provide a thorough understanding of the present technology. However, it will be apparent to one skilled in the art that the present technology may be practiced in other aspects that depart from these specific details.

The accompanying drawings, where like reference numerals refer to identical or functionally similar elements throughout the separate views, together with the detailed description below, are incorporated in and form part of the specification, and serve to further illustrate aspects of concepts that include the claimed disclosure and explain various principles and advantages of those aspects.

The electronic medical devices disclosed herein have been represented where appropriate by conventional symbols in the drawings, showing only those specific details that are pertinent to understanding the various aspects of the present disclosure so as not to obscure the disclosure with details that will be readily apparent to those of ordinary skill in the art having the benefit of the description herein.

FIG. 1 depicts an electronic medical device that includes one or more than one wireless electrical actuator (e.g., actuation button) to control the functions of the medical device, according to at least one aspect of the present disclosure.

FIG. 2 illustrates a circuit that utilizes mutual inductance M between a primary inductor and a secondary inductor, in accordance with at least one aspect of the present disclosure.

FIG. 3 illustrates a circuit that utilizes mutual inductance M between a primary inductor and a secondary inductor, in accordance with at least one aspect of the present disclosure.

FIG. 4 illustrates a circuit that utilizes mutual inductance M between a primary circuit and a secondary circuit, in accordance with at least one aspect of the present disclosure.

FIG. 5 illustrates a circuit that detects the state of a secondary circuit by utilizing mutual inductance M and a frequency-swept input signal, in accordance with at least one aspect of the present disclosure.

FIG. 6 illustrates a circuit that detects the state of multiple circuits in proximity to a primary circuit, in accordance with at least one aspect of the present disclosure.

FIG. 7 illustrates a circuit that detects the state of multiple secondary circuits in proximity to multiple primary circuits, in accordance with at least one aspect of the present disclosure.

FIG. 8 depicts an electronic medical device, in accordance with at least one aspect of the present disclosure.

FIG. 9 depicts circuit including a primary circuit and a secondary circuit, in accordance with at least one aspect of the present disclosure.

FIG. 10 depicts a primary circuit, in accordance with at least one aspect of the present disclosure.

FIG. 11 shows a pair of external buttons designed for a medical device, in accordance with at least one aspect of the present disclosure.

FIG. 12A presents a side elevation section view of the first button, highlighting its internal features, in accordance with at least one aspect of the present disclosure.

FIG. 12B offers a plan view of the first button without the dome, highlighting the internal features of the button, in accordance with at least one aspect of the present disclosure.

### DESCRIPTION

The present disclosure describes a parasitic electromagnetic (EM) field electrical actuator designed to transmit signals across a barrier to a sealed, reusable electronic module within a medical device. This actuator, such as an activation button, features an inductive coil, a resistor, and a switch. It draws power only upon activation, ensuring no power consumption when inactive.

The wireless parasitic EM field electrical actuator transmits signals across a barrier and features an isolated parasitic switch with a circuit comprising an inductive coil and a resistive element. When the switch is mechanically closed, the resistor connects to the coil, enabling signal transfer across the sealed barrier.

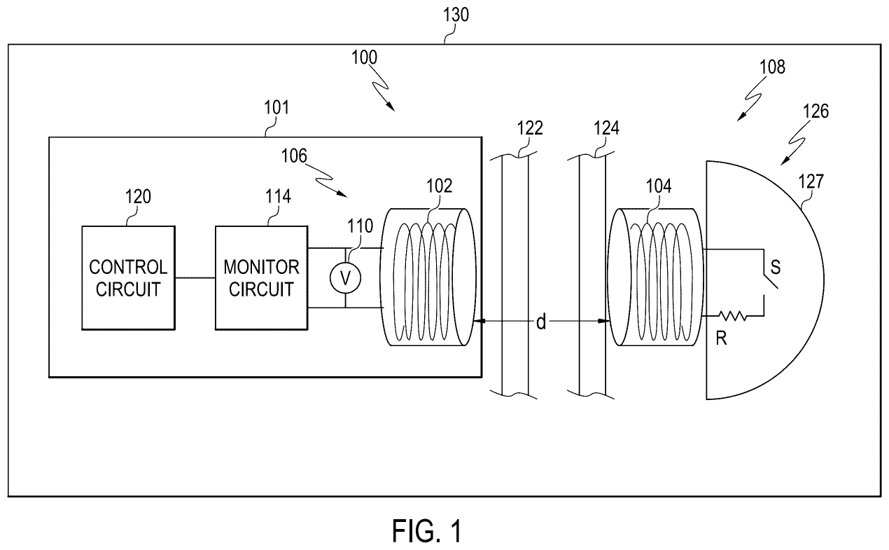

Turning to the figures, FIG. 1 depicts an electronic medical device 130 that includes one or more than one wireless electrical actuator 126 (e.g., actuation button) to control the functions of the medical device 130, according to at least one aspect of the present disclosure. In one aspect, the medical device 130 includes an electrical actuator 126 positioned on an exterior wall 124 of the medical device housing. The electrical actuator 126 includes an elastomer 127 and can be affixed to the exterior wall 124 of the medical device 130 using an adhesive. The electrical actuator 126 features a parasitic EM field switch S that can be sensed wirelessly by an electronic control circuit 120 positioned within a sealed housing 122 of the medical device 130. Thus, the activation of the electrical actuator 126 by a user can be sensed across the sealed housing 122 barrier allowing for the sterilization or autoclaving and reuse of the electronic components disposed within the sealed housing 122 barrier and the disposal of the electrical actuator 126 along with the medical device 130 housing.

An electronic control module 101 disposed within the sealed replaceable housing 122 includes a primary circuit 106 that generates a coupled inductance field between an internal inductor 102 disposed within the sealed housing 122 and an external inductor 104 of a secondary circuit 108 positioned on the exterior wall 124 of the medical device 130, making the electrical actuator 126 accessible to the user. The inductors 102, 104 are positioned in proximity to each other at a distance d, which is carefully selected to ensure adequate coupling of the EM field between them. The mutual inductance M is determined as a function of the distance d between the centers of two inductors 102, 104 from the frequency dependence of the real and imaginary parts.

The external inductor 104 of the electrical actuator 126 does not consume power when the electrical actuator 126 is not pressed and the switch S is open, remaining in an open loop configuration. When the electrical actuator 126 is pressed, the switch S closes to complete the loop, allowing electrical flow, e.g., inductively coupled current, through the inductor 104 and the resistor R in the secondary circuit 108.

Thus, when the elastomer 127 portion of the electrical actuator 126 is pressed and the switch S is closed, mutual inductance M couples energy from the primary circuit 106 to the secondary circuit 108, enabling electrical flow in the secondary circuit 108. This allows a monitor circuit 114 coupled to the control circuit 120 in the primary circuit 106 to detect user activation of the electrical actuator 126 to control a function of the medical device 130.

In operation, when power is applied by a power source 110 a large inrush current flows into the primary inductor 102, which then drops to near zero as the magnetic field inhibits further current flow. When the switch S is closed and the secondary inductor 104 is in a closed loop configuration, the secondary inductor 104 draws power from the field established by the primary inductor 102. The resulting current flowing in the primary circuit 106 increases to re-saturate the field. Once re-saturated, and as long as the secondary coil is not consuming any of the field, the current should drop to zero again.

The electrical actuators 126 disposed on an exterior wall of the medical device 130 do not require externally applied power, are inexpensive, and contain no integrated circuit electronic components, thus making their disposal convenient and economical. The integration of the passive electrical components such as the external inductor 104, and other tunable elements such as capacitors, into the electrical actuator 126 allows for a variety of switch sizes, shapes, and user feel options.

We now proceed to describe the operation of a mutual inductance circuit. The analysis of a mutual inductance circuit involves understanding the interaction between the inductors, setting up the appropriate differential equations, and solving them using standard circuit analysis techniques. The effects of mutual inductance are significant in many practical applications, especially in the design of transformers and coupled inductors.

Mutual inductance in circuits occurs when two or more inductors are magnetically coupled, meaning the magnetic field of one inductor induces a voltage across the other. This phenomenon is fundamental in transformers, coupled inductors, and some types of filters. Below is a description of the analysis process for a mutual inductance circuit.

The circuit configuration of a mutual inductance circuit usually consists of at least two coils or inductors, L1 and L2, with mutual inductance M between them and a coupling coefficient k. The coupling coefficient, k, describes the extent of the magnetic coupling between the inductors and is defined as:

$$M = k \sqrt{{L}_{1} {L}_{2}}$$

where 0≤k≤1.

The voltage induced in each inductor, L1 and L2, are described by Faraday's law. For the two inductors L₁ and L₂, the voltage equations can be written as:

$${V}_{1} = {L}_{1} \frac{{dI}_{1}}{d t} + M \frac{{dI}_{2}}{d t}$$

$${V}_{2} = {L}_{2} \frac{{dI}_{2}}{d t} + M \frac{{dI}_{1}}{d t}$$

where:
   V₁ and V₂ are the voltages across L₁ and L₂, respectively.
   I₁ and I₂ are the currents through L₁ and L₂, respectively.
   M is the mutual inductance.

Depending on the complexity of the mutual inductance circuit, various methods can be used for analysis. Mesh analysis may be applied to the loops in the circuit, incorporating the mutual inductance M terms into the voltage equations. Nodal analysis may be applied if there are nodes where the inductors connect, nodal analysis might be more appropriate. Alternating current (AC) steady-state analysis may be used if the circuit is driven by an AC source, a phasor analysis to solve for the sinusoidal steady-state currents and voltages.

The energy stored in the inductors L₁ and L₂, considering mutual inductance M, is given by:

$$W = \frac{1}{2} {L}_{1} {I}_{1}^{2} + \frac{1}{2} {L}_{2} {I}_{2}^{2} + {MI}_{1} {I}_{2}$$

If the circuit forms an LC circuit with capacitances, the resonant frequency can be affected by the mutual inductance M.

In transformers, the mutual inductance M is key to how impedances are transformed between the primary and secondary windings.

Practical considerations include leakage inductance because not all the magnetic flux from one inductor links to the other. The inductance not contributing to mutual coupling is the leakage inductance. Additionally, real-world losses can be due to the resistance of the coils and core losses in magnetic materials.

The following is an example where two inductors L₁=1 H and L₂=2 H are coupled with a mutual inductance M=0.5 H. Suppose I₁(t)=cos ωt is the current in the first inductor L₁, and you need to find the voltage across the second inductor L₂.

Using the voltage equation for L₂:

$${V}_{2} ( t ) = {L}_{2} \frac{{dI}_{2}}{d t} + M \frac{{dI}_{1}}{d t}$$

To calculate the derivative of I₁(t), substitute the values, and solve for V₂(t).

Below is a description of one embodiment of a mutual inductance-based circuit in which a direct current (DC) voltage is applied to the input circuit, and a switch is inserted in the output circuit. This embodiment illustrates the concept of using mutual inductance with a DC voltage source and a switch in the output circuit to control the transfer of energy or the induced voltage in the secondary circuit. Additional aspects, including the type of switch, configurations, and other circuit elements, are also disclosed.

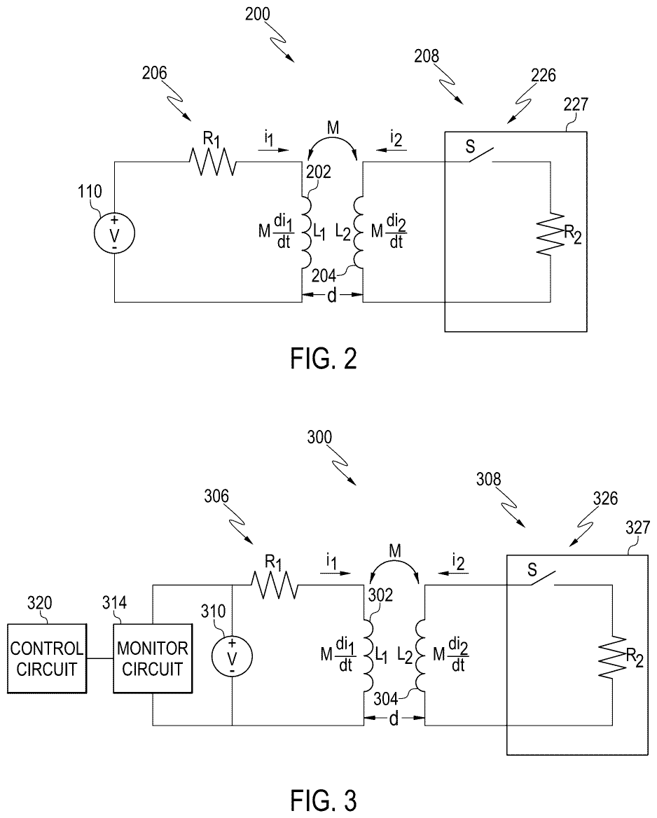

Referring to FIG. 2 a circuit 200 is illustrated that utilizes mutual inductance M between a primary inductor 202 and a secondary inductor 204, in accordance with at least one aspect of the present disclosure. The circuit 200 includes a primary circuit 206 and a secondary circuit 208. The primary circuit 206 includes a power source 210, which may be a DC voltage source or an AC voltage source, and a primary inductor 202 with inductance L₁ connected to the power source 210, The primary inductor 202 generates a magnetic field in response to the applied DC voltage V_{DC}.

The secondary circuit 208 is magnetically coupled to the primary inductor 202. The secondary circuit 208 includes a secondary inductor 204 with inductance L₂ arranged in proximity to the primary inductor 202 to induce a voltage V_{M} via mutual inductance M and a switch S connected in series with the secondary inductor 204. The inductors 202, 204 are positioned in close proximity to each other at a distance d, which is carefully selected to ensure adequate coupling of the EM field between them. The mutual inductance M is determined as a function of the distance d between the centers of two inductors 202, 204 from the frequency dependence of the real and imaginary parts.

The switch S is operable to control the flow of current I_{L2} through the secondary inductor 204 based on the induced voltage V_{M}. The magnetic coupling between the primary inductor 202 and the secondary inductor 204 is characterized by a mutual inductance M such that the switch S enables or disables energy transfer from the primary inductor 202 to the secondary inductor 204, to control the induced voltage V_{M} or current I_{L2} in the secondary circuit 208.

The switch S and a tuning element, such as a resistor, inductor, or capacitor, are integrated into an electrical actuator 226 (e.g., actuation button) on the medical device, which activates a predefined function. In one aspect, the electrical actuator 226 is encased in an elastomer 227 and can be affixed to the exterior wall of the medical device using an adhesive. When the user activates the electrical actuator 226 by pressing on it, for example, the switch S closes, and through mutual inductance M, the secondary circuit 208 detunes the primary circuit 206, triggering a detectable event by the monitor circuit as described below.

Below is a description of another embodiment of a mutual inductance-based circuit to monitor the electrical characteristics across the primary inductor before and after the switch in the secondary circuit is closed. This embodiment is directed to the dynamic interaction between the circuits during switching events. This embodiment introduces a monitor circuit to detect a change in the electrical characteristics of the primary circuit, including changes in voltage, current, impedance, frequency, or phase, or combinations thereof, based on the state of the switch (open or closed). In one aspect, the monitor circuit measures and compares the voltage across the primary inductor to detect changes in the primary circuit caused by the closing of the switch in the secondary circuit.

Referring to FIG. 3, a circuit 300 is illustrated that utilizes mutual inductance M between a primary inductor 302 and a secondary inductor 304, in accordance with at least one aspect of the present disclosure. The circuit 300 includes a primary circuit 306 and a secondary circuit 308. The primary circuit 306 includes a power source 310, which may be a DC voltage source or an AC voltage source, and a primary inductor 302 with inductance L₁ connected to the power source 310. The primary inductor 302 generates a magnetic field in response to the applied voltage V.

In one aspect, a monitor circuit 314 measures and compares the voltage V_{L1} across the primary inductor 302 and detect any changes based on the open or closed state of the switch S. In other aspects, the monitor circuit 310 measures and compares the current, impedance, frequency, or phase, or combinations thereof, based on the open or closed state of the switch S.

The secondary circuit 308 is magnetically coupled to the primary inductor 302. The secondary circuit 308 includes a secondary inductor 304 with inductance L₂ arranged in proximity to the primary inductor 302 to induce a voltage V_{M} via mutual inductance M. The inductors 302, 304 are positioned in close proximity to each other at a distance d, which is carefully selected to ensure adequate coupling of the EM field between them. The mutual inductance M is determined as a function of the distance d between the centers of two inductors 302, 304 from the frequency dependence of the real and imaginary parts.

The switch S is connected in series with the secondary inductor 304. The switch S is operable to control the flow of current I_{L2} through the secondary inductor 304 in response to the induced voltage V_{M}. In one aspect, the monitor circuit 314 measures and compares the voltage V_{M} across the primary inductor 302 both before and after the switch S in the secondary circuit 308 is closed, enabling detection of changes in the primary circuit's voltage V_{L1} due to the mutual inductive coupling when the switch S is engaged to enable the assessment of the impact of the secondary circuit 308 on the operation of the primary circuit 306. In other aspects, the monitor circuit 310 measures and compares the current, impedance, frequency, or phase, or combinations thereof, based on the open or closed state of the switch S.

The switch S and a tuning element, such as a resistor, inductor, or capacitor, are integrated into an electrical actuator 326 (e.g., actuation button) positioned on an exterior wall of a medical device. The electrical actuator 326 activates a predefined function of the medical device. In one aspect, the electrical actuator 326 is encased in an elastomer 327 and can be affixed to the exterior wall of the medical device using an adhesive. When the user depresses the electrical actuator 326, the switch S closes, and through mutual inductance M, the secondary circuit 308 detunes the primary circuit 306, triggering a detectable event by the monitor circuit 314.

Below is a description of yet another embodiment of a mutual inductance-based circuit featuring coupled primary and secondary circuits. This embodiment enables the monitoring of a switch in the secondary circuit by observing changes in electrical characteristics in the primary circuit. The monitor circuit detects changes in resonance, which serve as clear indicators of a switch closure, making this configuration highly useful for sensing and control applications. The monitor circuit can detect changes in resonance based on the state of the switch (open or closed) and any resulting variations in the electrical characteristics of the primary circuit. These variations in the electrical characteristics of the primary circuit may include changes in voltage, current, impedance, frequency, phase, or combinations thereof.

The circuit includes coupled primary and secondary circuits featuring the mutual inductive coupling between the primary and secondary circuits, the role of a switch in the secondary circuit, and the ability of a monitor circuit to detect and analyze the state of the system based on the observed voltage changes in the primary circuit.

Additionally, an analysis of the circuit is provided, detailing the primary and secondary circuits coupled by mutual inductance. The monitor circuit observes the primary circuit to detect the switch closure in the secondary circuit, highlighting the interaction and the utility of this setup in detecting and analyzing switching events.

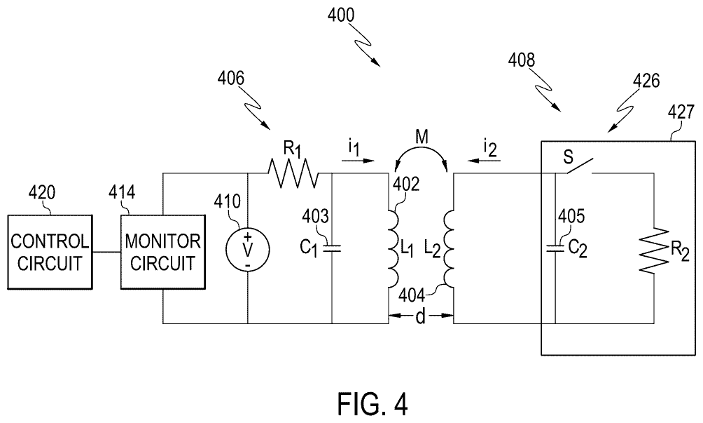

Turning to FIG. 4, a circuit 400 is illustrated that utilizes mutual inductance M between a primary circuit 406 and a secondary circuit 408, in accordance with at least one aspect of the present disclosure. The circuit 400 includes two circuits—a primary circuit 406 and a secondary circuit 408—that are magnetically coupled through mutual inductance M when the loop in the secondary circuit 408 is closed by a switch S. Both circuits 406, 408 resonates at a specific frequency, determined by their respective inductances L₁, L₂ and capacitances C₁, C₂.

A monitor circuit 414 is placed in the primary circuit 406 to detect changes in the electrical characteristics of the primary circuit 406 based on a change of state (open or closed) of the switch S in the secondary circuit 408. In various aspects, the monitor circuit 414 can detect changes in resonance in the primary circuit 406 based on the state of the switch S (open or closed) and any resulting variations in the electrical characteristics of the primary circuit 406. These variations may include changes in voltage, current, impedance, frequency, phase, or combinations thereof.

The circuit 400 utilizes mutual inductance M for energy transfer between coupled primary and secondary circuits 406, 408 and monitoring the primary circuit 406. The circuit 400 includes a primary circuit 406 and secondary circuit 408.

The primary circuit 406 includes an inductor 402 with inductance L₁ and a capacitor 403 with capacitance C₁ connected in series or parallel to form the primary circuit 406 with a resonant frequency f₁. A power source 410 supplies electrical energy to the primary circuit 406. In one aspect, the monitor circuit 414 is operatively connected to the primary circuit 406 measures the voltage across the primary circuit 406. The power source 410 may be a DC voltage source or an AC voltage source.

A secondary circuit 408 is positioned in proximity of the primary circuit 406 to enable magnetic coupling through mutual inductance M between the primary and secondary circuits 406, 408. The secondary circuit 408 includes an inductor 404 with inductance L₂ and a capacitor 405 with capacitance C₂ connected in series or parallel to form the secondary circuit 408 with a resonant frequency f₂. The inductors 402, 404 are positioned in close proximity to each other at a distance d, which is carefully selected to ensure adequate coupling of the EM field between them. The mutual inductance M is determined as a function of the distance d between the centers of two inductors 402, 404 from the frequency dependence of the real and imaginary parts.

The switch S is connected in series or parallel with the secondary circuit 408. The switch S controls the activation of the secondary circuit 408.

In operation, the primary circuit 406 and the secondary circuit 408 are magnetically coupled based on the state of the switch S (open or closed) to induce voltage or current in the secondary circuit 408 by mutual inductance M in response to the operation of the primary circuit 406. Activation of the switch S in the secondary circuit 408 influences the electrical characteristics in the primary circuit 406. The influence on the electrical characteristics in the primary circuit 406 may be detected by the monitor circuit 414 as a change in voltage, current, impedance, frequency, or phase, or combinations thereof.

In one aspect, the monitor circuit 414 in the primary circuit 406 detects changes in the voltage across the primary circuit 406 resulting from the mutual inductive coupling between the primary and secondary circuits 406, 408. A measurable change in the voltage response of the primary circuit 406 occurs when the secondary circuit 408 is activated by the closure of the switch S. The monitor circuit 414 provides an indication of the state of the secondary circuit 408 based on the detected voltage changes in the primary circuit 408. Accordingly, the circuit 400 enables detection, monitoring, and analysis of the interaction between the primary and secondary circuits 406, 408 through mutual inductance S and the state of the switch S.

Below is a description of the primary and secondary circuits 406, 408. Resonance of the secondary circuit 408 depends on the open/closed state of the switch S. For the sake of brevity, the second resonant circuit 408 is referred to as “resonant circuit” whether it is in resonance or not in resonance based on the open/closed state of the switch S.

The primary circuit 406 includes an inductor 402 (L₁), a capacitor 405 (C₁), a power source 410, which can be a DC V(DC) or AC voltage source v(AC), and a monitor circuit 414 across L₁ and C₁. The natural resonant frequency f₁ of the primary circuit 406 is given by:

$${f}_{1} = \frac{1}{2 π \sqrt{{L}_{1} {C}_{1}}}$$

The monitor circuit 414 is located in the primary circuit 406 to measure the voltage across the primary circuit 406, specifically across L₁ or C₁, to observe changes when mutual inductance M comes into play.

The secondary circuit 408 includes an inductor 404 (L₂), a capacitor 405 (C₂), and a switch S. The natural resonant frequency f₂ of the secondary circuit 408 is given by:

$${f}_{2} = \frac{1}{2 π \sqrt{{L}_{2} {C}_{2}}}$$

The circuit 400 may be characterized by mutual Inductance M and coupling coefficient k. The mutual inductance M between inductors L₁ and L₂ causes the magnetic field generated by the current in the primary circuit 406 to induce a voltage in the secondary circuit 408.

The coupling coefficient k is defined as:

$$M = k \sqrt{{L}_{1} {I}_{2}}$$

The strength of the coupling affects how much energy is transferred between the primary and secondary circuits 406, 408.

The operation and monitoring of changes in the electrical characteristics of the primary circuit 406 involve analyzing the conditions with the switch S open and the switch S closed in the secondary circuit 408.

When the switch S in the secondary circuit 408 is open, the secondary circuit 408 is not in resonance, and no significant current flows through it. The primary circuit 406 resonates at its natural frequency f₁, and the monitor circuit 414 observes a steady-state condition corresponding to this resonant condition. The electrical characteristics in the primary circuit 406 will reflect normal resonant behavior, with the amplitude depending on the Q-factor (quality factor) of the primary circuit 406. As discussed above, the electrical characteristics include, without limitation, voltage, current, impedance, frequency, or phase, or combinations thereof.

The switch S and a tuning element, such as a resistor, inductor, or capacitor 405, are integrated into an electrical actuator 426 on the medical device, which activates a predefined function. In one aspect, the electrical actuator 426 is encased in an elastomer 427 and can be affixed to the exterior wall of the medical device using an adhesive. When the user depresses the electrical actuator 426, the switch S closes, and through mutual inductance M, the secondary circuit 408 detunes the primary circuit 406, triggering a detectable event by the monitor circuit 414.

In the embodiment illustrated in FIG. 4, when the switch S is closed, the secondary circuit 408 forms a resonant loop. If the secondary resonant frequency f₂ is close to the primary resonant frequency f₁, strong coupling will occur, and energy will transfer between the resonant circuits 406, 408 via mutual inductance M. This coupling leads to a change in the current distribution in both resonant circuits 406, 408, causing a shift in the resonant conditions.

The closing of the switch S in the secondary circuit 408 alters the magnetic coupling, causing a change in the electrical characteristics in the primary circuit 406. For example, in one aspect, the change in the electrical characteristics is a change in voltage across L₁ and C₁ in the primary circuit 408. The monitor circuit 414 in the primary circuit 406 detects this change, which manifests as a shift in amplitude, phase, or both. The exact nature of the change depends on factors such as the coupling strength k, the resonant frequencies f₁ and f₂, and the relative phase of the currents in both resonant circuits 406, 408.

The behavior of the coupled resonant circuits 406, 408 can be described by the following differential equations:

$${V}_{D C} = {L}_{1} \frac{{dI}_{1}}{d t} + \frac{1}{{C}_{1}} \int {I}_{1} d t + M \frac{{dI}_{2}}{d t}$$

$$0 = {L}_{2} \frac{{dI}_{2}}{d t} + \frac{1}{{C}_{2}} \int {I}_{2} d t + M \frac{{dI}_{1}}{d t} + ( Switch Effects )$$

Here, I₁ and I₂ are the currents in the primary and secondary circuits 406, 408, respectively. The monitor circuit 414 is sensitive to the term

$$M \frac{{dI}_{2}}{d t} ,$$

which represents the effect of the secondary circuit 408 on the primary circuit 408.

The responses of the resonant circuits 406, 408 can be analyzed in the frequency domain by considering the impedance of the resonant circuits 406, 408 and how the mutual inductance M affects the total impedance seen by the primary circuit 406. The presence of mutual inductance M modifies the resonance conditions, which will be detected as a shift in the voltage monitored by the monitor circuit 414.

The closing of the switch S in the secondary circuit 408 can cause a detuning of the primary circuit 406, resulting in a noticeable shift in the resonant frequency f₁ and voltage levels observed by the monitor circuit 414.

The energy transferred between the primary and secondary circuits 406, 408 is maximized when f₁≈f₂. The monitor circuit 414 can thus detect the resonance conditions and confirm switch S operation in the secondary circuit 408.

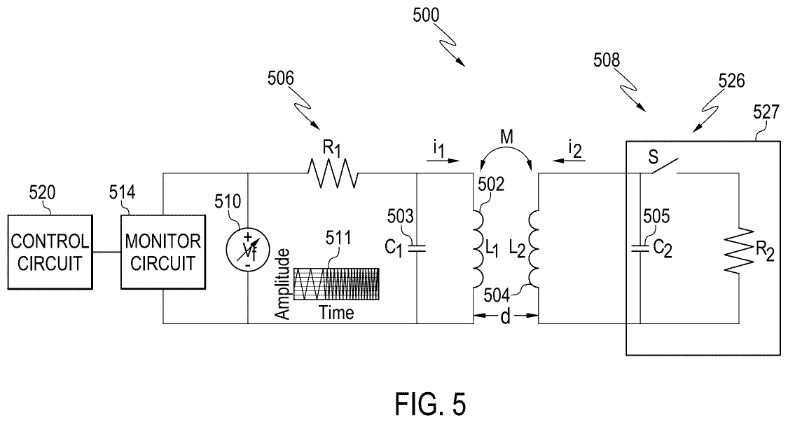

Turning to FIG. 5, a circuit 500 is illustrated that detects the state of a secondary circuit 508 by utilizing mutual inductance M and a frequency-swept input signal 511, in accordance with at least one aspect of the present disclosure. The circuit 500 includes a primary circuit 506. The primary circuit 506 includes an inductor 502 with inductance L₁ and a capacitor 503 with capacitance C₁ to form a primary resonant circuit with a resonant frequency f₁.

A frequency-swept AC signal source 510 is connected to the primary circuit 506. The signal source 510 generates a signal 511 whose frequency varies over a predetermined range of frequencies that includes the resonant frequency f₁. In one aspect, the frequency sweep range is 1 Hz to 50,000 Hz, for example.

A monitor circuit 514 is connected to the primary circuit 506. The monitor circuit 514 detects changes in the electrical characteristics of the primary circuit 506 over the frequency range of the swept input signal 510 based on the state of a switch S (open or closed) in a secondary circuit 508.

A secondary circuit 508 is magnetically coupled to the primary circuit 506 through mutual inductance M. The secondary circuit 508 includes an inductor 504 with inductance L₂ and a capacitor 505 with capacitance C₂ to form a secondary resonant circuit with a resonant frequency f₂.

The secondary circuit 508 includes a switch S connected in series or in parallel with the inductor 504 and capacitor 505. The switch S controls the activation of the secondary circuit 508.

The mutual inductance M between the primary circuit 506 and the secondary circuit 508 causes a detectable change in the electrical characteristics or response of the primary circuit 506 as measured by the monitor circuit 514. For example, when the switch S in the secondary circuit 508 is open, the monitor circuit 514 detects a standard resonant response at f₁. When the switch S in the secondary circuit 508 is closed, the monitor circuit 514 detects a modified electrical characteristic response in the primary circuit 506 characterized by shifts, splits, or anomalies in the resonance curve due to the coupling with the secondary circuit at f₂.

In one aspect, selecting a value of the tuning element in the secondary circuit 508. For example, the tuning element may be the inductance L₂ of the inductor 504 and/or the capacitance C₂ of the capacitor 505 to produce a resonant frequency f₂ that falls within the range of frequencies swept by the signal source 510. This enables the monitor circuit 514 to detect the selection of more than one electrical actuator 526 (e.g., actuation button) disposed on the medical device 100 (FIG. 1). Accordingly, if the monitor circuit 514 is to detect two or more electrical actuator 526 disposed on the medical device 100, each electrical actuator 526 would have a unique combination of tuning elements to tune the secondary circuit 508 to a different resonant frequency that falls with a resonant frequency of frequencies swept by the signal source 510.

The state of the switch S in the secondary circuit 508 is inferred by analyzing the electrical characteristics response of the primary circuit 506 during the frequency sweep.

Below is a description of the mutual-inductance circuit 500 utilizing a power source 510 to generate frequency-swept AC signal 511 as an input to the resonant circuit formed by inductor 502 with inductance L₁ and a capacitor 503 with capacitance C₁ in the primary circuit 506. Applying the frequency-swept AC signal 510 in the primary circuit 506 allows for dynamic analysis of the system's response. The mutual inductance M between the primary and secondary circuits 506, 508 introduces frequency-dependent behavior, significantly influenced by the state of the switch S in the secondary circuit 508. The observations of the monitor circuit 514 during the frequency sweep reveal the interaction between the two circuits, providing insights into the resonance conditions and the effects of coupling. This setup is highly useful for sensing, detection, and signal processing applications.

The circuit 500 shown in FIG. 5 uses the frequency-swept AC signal 511 applied in the primary circuit 506 to monitor changes in the in electrical characteristics (e.g., voltage, current, frequency, phase, amplitude, etc.) response caused by mutual inductance M with the secondary circuit 508. The presence or absence of anomalies in the response indicates the state of the switch S in the secondary circuit 508, providing a method for detecting and analyzing the interaction between the two circuits 506, 508 when they are in resonance.

When the frequency-swept AC signal 511 is applied in the primary circuit 506, the behavior of the circuit 500 changes dynamically across a range of frequencies. This approach allows the analysis of how the mutual inductance M between the primary and secondary circuits 506, 508 affects the overall response, particularly when the switch S in the secondary circuit 508 is opened or closed.

The primary circuit 506 is driven by the frequency-swept AC signal 511 generated by the frequency-swept AC source 510, meaning the input signal's frequency gradually changes over a specified range. The secondary circuit 508, magnetically coupled to the primary circuit 506, can affect the response of the primary circuit 506, particularly near resonant frequencies.

The resonance conditions of the primary circuit 506 is based on the inductance L₁ and capacitance C₁, which form the resonant circuit. The resonant frequency f₁ of the primary circuit 506 is given by:

$${f}_{1} = \frac{1}{2 π \sqrt{{L}_{1} {C}_{1}}}$$

The secondary circuit 508 resonance is based on the inductance L₂ and capacitance C₂, which form the secondary circuit 508. The resonant frequency f₂ of the secondary circuit 508 is given by:

$${f}_{2} = \frac{1}{2 π \sqrt{{L}_{2} {C}_{2}}}$$

Below is a discussion of the frequency response of the primary circuit 506 based on the state of the switch S (open or closed). The primary circuit 506 is excited by an AC power source 510, which generates a frequency swept signal 511 that sweeps over a range of frequencies that includes f₁ and potentially f₂. As the frequency of the AC signal 511 approaches f₁, the primary circuit 506 will exhibit a peak in voltage or current due to resonance.

Mutual inductance M between L₁ and L₂ causes energy transfer between the primary and secondary circuits 506, 508. If the secondary circuit 508 is open (switch S open), it may have little effect on the primary circuit 506 unless there is some residual coupling or capacitance. If the switch S in the secondary circuit 508 is closed, the secondary circuit 508 will resonate at f₂, influencing the response of the primary circuit 506.

Below is a description of the impact of the switch S in the secondary circuit 508 on the response of the primary circuit 506 when the switch S is open and the secondary circuit 508 is inactive and when the switch S closed and the secondary circuit 508 is active.

When the switch S is open, the secondary circuit 508 is inactive. In other words, with the switch S open, the secondary circuit 508 does not form a closed loop, resulting in minimal mutual interaction. The primary circuit 506 exhibits a typical resonant peak at f₁ when the frequency sweep passes through its resonant frequency. The monitor circuit 514 would observe a standard resonance curve with no additional anomalies.

When the switch S is closed, the secondary circuit 508 becomes active. The switch S and a tuning element, such as a resistor, inductor, or capacitor 505, are integrated into the electrical actuator 526, which is disposed on the medical device to activate a predefined function. In one aspect, the electrical actuator 526 is encased in an elastomer 527 and can be affixed to the exterior wall of the medical device using an adhesive. When the user depresses the electrical actuator 526, the switch S closes, and through mutual inductance M, the secondary circuit 508 detunes the primary circuit 506, triggering a detectable event by the monitor circuit 514.

In the embodiment illustrated in FIG. 5, upon closing the switch S, the secondary circuit becomes active and can resonate at its natural frequency f₂. If f₁≈f₂, significant mutual inductive coupling occurs, leading to energy exchange between the two circuits. This coupling modifies the impedance of the primary circuit to alter its resonant peak.

The voltage across the primary circuit 506, monitored during the frequency sweep, will show changes when the switch S is closed and the secondary circuit 508 becomes active. These changes may manifest as split resonance peaks, a shift in response, or the presence of anomalies.

Split resonance peaks occur if the primary and secondary circuits 506, 508 are strongly coupled (with f₁ close to f₂). In this scenario, the primary circuit 506 may exhibit a “split” in the resonance peak, showing two distinct peaks corresponding to the coupled resonant modes.

A shift in resonance can occur depending on the coupling strength and the difference in resonant frequencies. The resonant peak in the primary circuit 506 may shift, reduce in amplitude, or broaden.

Near the resonant frequency f₂ of the secondary circuit 508, the primary circuit 506 might show a dip or anomaly in its voltage response due to energy being siphoned off into the secondary circuit 508.

A quantitative analysis necessitates examining coupled differential equations. The coupled behavior can be described by a set of linear differential equations:

$${V}_{i n} ( t ) = {L}_{1} \frac{{dI}_{1}}{d t} + \frac{1}{{C}_{1}} \int {I}_{1} d t + M \frac{{dI}_{2}}{d t}$$

$$0 = {L}_{2} \frac{{dI}_{2}}{d t} + \frac{1}{{C}_{2}} \int {I}_{2} d t + M \frac{{dI}_{1}}{d t} + ( Switch Effects )$$

Solving these equations in the frequency domain (using phasor analysis) gives the impedance and voltage as functions of frequency.

From an impedance perspective, the total impedance Zₜₒₜₐₗ seen by the AC source 510 driving the primary circuit 506 is affected by the mutual inductance M and the state of the secondary circuit 508. The impedance Z can be expressed as:

$${Z}_{total} ( ω ) = {Z}_{1} ( ω ) + \frac{{M}^{2}}{{Z}_{2} ( ω )}$$

Here, Z₁(ω) and Z₂(ω) are the impedances of the primary and secondary circuits 506, 508, respectively, at angular frequency ω.

The monitor circuit 514 has a baseline resonance when the switch S open, detecting a single peak at f₁, which corresponds to the natural resonance of the primary circuit 506.

When the switch S is closed, the secondary circuit 508 becomes active, leading to distorted resonance. The monitor circuit 514 may then detect multiple peaks, peak splitting, or a shifted resonance, depending on the coupling and resonance conditions.

The shape and behavior of the curve detected during the frequency sweep provide essential information about the coupling strength, the resonant frequency of the secondary circuit 508, and the state of the switch S.

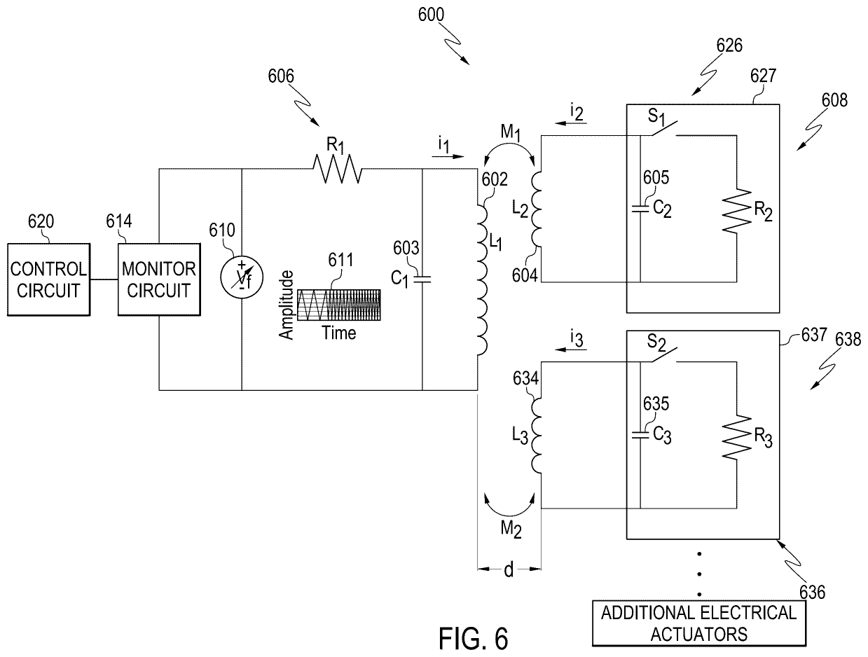

Turning to FIG. 6, a circuit 600 is illustrated that detects the state of multiple circuits in proximity to a primary circuit, in accordance with at least one aspect of the present disclosure. In this embodiment, the primary circuit 606 is connected to nearby circuits, such as a secondary circuit 608 and a tertiary circuit 638, using mutual inductances M₁ and M₂, along with a frequency-swept input signal 611. The primary circuit 606 features an inductor 602 with inductance L₁ and a capacitor 603 with capacitance C₁, forming a primary resonant circuit with a resonant frequency f₁.

The primary circuit 606 features an inductor 602 with inductance L₁ and a capacitor 603 with capacitance C₁, forming a primary resonant circuit with a resonant frequency f₁. The primary circuit 606 includes a frequency-swept AC signal source 610 connected to the resonant circuit formed by the inductor 602 and capacitor 603. The signal source 610 generates a frequency-swept signal 611, varying over a range that includes the resonant frequency f₁. The frequency sweep range can be from 1 Hz to 50,000 Hz, for example.

A monitor circuit 614 is coupled to the primary circuit 606. The monitor circuit 614 detects changes in the electrical characteristics of the primary circuit 606 across the frequency range of the swept input signal 610. These changes are based on the state (open or closed) of a first switch S₁ in a secondary circuit 608 or a second switch S₂ (open or closed) in a tertiary circuit 638.

A first secondary circuit 608 is magnetically coupled to the primary circuit 606 through mutual inductance M₁ when the inductor 602 is energized. The first secondary circuit 608 includes an inductor 604 with inductance L₂ and a capacitor 605 with capacitance C₂, forming a first secondary resonant circuit with a resonant frequency f₂. The first secondary circuit 608 also features an electrical actuator 626, comprising an elastomer 627 mechanically linked to a switch S₁. This switch S₁ is connected in series or parallel with the inductor 604 and the capacitor 605, regulating electrical flow in the first secondary circuit 608. When the switch S₁ is open, no electricity flows, leaving the first secondary circuit 608 unactuated. When the switch S₁ is closed, electricity flows, activating the first secondary circuit 608. Additional electrical actuators can be added to the circuit 600.

A second secondary circuit 638 is magnetically coupled to the primary circuit 606 through mutual inductance M₂ when the inductor 602 is energized. The second secondary circuit 638 includes an inductor 634 with inductance L₃ and a capacitor 635 with capacitance C₃, forming a second secondary resonant circuit with a resonant frequency f₃. The second secondary circuit 638 also features an electrical actuator 636, comprising an elastomer 637 mechanically linked to a switch S₂. This switch S₂ is connected in series or parallel with the inductor 634 and the capacitor 635, regulating electrical flow in the second secondary circuit 638. When the switch S₂ is open, no electricity flows, leaving the second secondary circuit 638 unactuated. When the switch S₂ is closed, electricity flows, activating the second secondary circuit 638. Additional electrical actuators can be added to the circuit 600.

The primary circuit 606 detects the actuation state of the second secondary circuits 608, 638. This is monitored by the monitor circuit 614, which communicates the state to the control circuit 620 within a sealed medical device housing. The control circuit 620 links the activation of the electrical actuators 626, 636 to specific device functions. User activation is sensed across the sealed barrier, allowing for sterilization and reuse of the internal components while enabling disposal of the actuators 626, 636 and housing.

The circuit 600 uses mutual inductance M for energy transfer between primary circuit 606 and the first and second secondary circuits 608, 638, and any additional secondary circuits coupled to the primary circuit 606. In one embodiment, the circuit 600 includes a second secondary circuit 638 positioned near the primary circuit 606, enabling magnetic coupling through mutual inductance M₂. The second secondary circuit 638 features a a second electrical actuator 636, which includes a third inductor 634 with inductance L₃ and a third capacitor 635 with capacitance C₃, forming a resonant circuit with frequency f₃. The second electrical actuator 636 activates the second secondary circuit 638.

The primary circuit 606 and the second secondary circuit 638 are magnetically coupled through the state of the electrical actuator 636. This induces electrical flow in the second secondary circuit 638 via mutual inductance M₂ when the inductor 602 in the primary circuit 606 is energized.

The monitor circuit 614 in the primary circuit 606 detects measurable changes in the primary circuit's 606 response due to mutual inductive coupling with the second secondary circuit 638. These changes are influenced by the state of the second electrical actuator 636, which depends on the actuation of the switch S₂ during the frequency sweep.

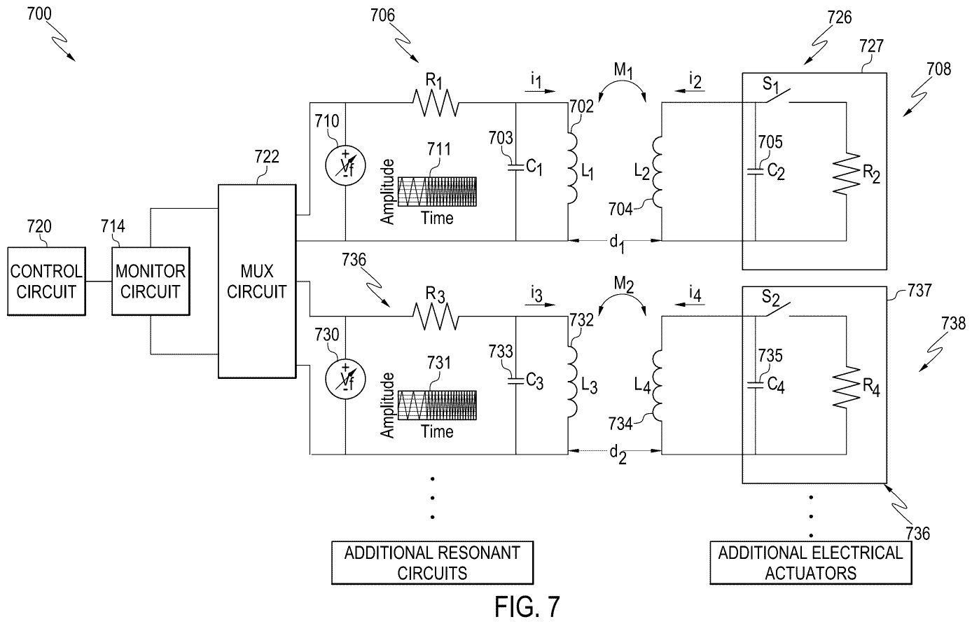

Turning to FIG. 7, a circuit 700 is illustrated that detects the state of multiple secondary circuits in proximity to multiple primary circuits, in accordance with at least one aspect of the present disclosure. In this embodiment, multiple primary circuits 706, 736 are positioned in proximity to multiple secondary circuits 708, 738, respectively. When the primary circuits 706, 736 are energized with frequency-swept input signals 711, 731 and the secondary circuits 708, 738 are actuated, the primary circuits 706, 736 are coupled to the secondary circuits 708, 738 through mutual inductances M₁, M₂. The deviation in the resonant circuits in the primary circuits 706, 736 due to the mutual inductance is detected by a monitor circuit 714, which communicates the actuation state of the secondary circuits 708, 738 to the control circuit 720 within a sealed medical device housing.

The first primary circuit 706 features an inductor 702 with inductance L₁ and a capacitor 703 with capacitance C₁, forming a first primary resonant circuit with a resonant frequency f₁. The second primary circuit 736 features an inductor 732 with inductance L₃ and a capacitor 733 with capacitance C₃, forming a second primary resonant circuit with a resonant frequency f₃. The first primary circuit 706 includes a first frequency-swept AC signal source 710 connected to the resonant circuit formed by the inductor 702 and capacitor 703. The second primary circuit 736 includes a second frequency-swept AC signal source 730 connected to the resonant circuit formed by the inductor 732 and capacitor 733. Each signal source 710, 730 generate frequency-swept signals 711, 731 varying over a range that includes the resonant frequencies f₁, f₃. The frequency sweep range can be from 1 Hz to 50,000 Hz, for example.

A monitor circuit 714 is coupled to the first and second primary circuits 706, 736 through a multiplexer 722. The monitor circuit 714 detects changes in the electrical characteristics of the first and second primary circuits 706, 736 across the frequency range of the swept input signals 711, 731. These changes are based on the state (open or closed) of a first switch S₁ in a first secondary circuit 708 or a second switch S₂ (open or closed) in a second secondary circuit 738.

A first secondary circuit 708 is magnetically coupled to the first primary circuit 706 through mutual inductance M₁ when the inductor 702 is energized. The first secondary circuit 708 includes an inductor 704 with inductance L₂ and a capacitor 705 with capacitance C₂, forming a first secondary resonant circuit with a resonant frequency f₂. The first secondary circuit 708 also features an electrical actuator 726, comprising an elastomer 727 mechanically linked to a switch S₁. This switch S₁ is connected in series or parallel with the inductor 704 and the capacitor 705, regulating electrical flow in the first secondary circuit 708. When the switch S₁ is open, no electricity flows, leaving the first secondary circuit 708 unactuated. When the switch S₁ is closed, electricity flows, activating the first secondary circuit 708. Additional electrical actuators can be added to the circuit 700.

A second secondary circuit 738 is magnetically coupled to the second primary circuit 736 through mutual inductance M₂ when the inductor 732 is energized. The second secondary circuit 738 includes an inductor 734 with inductance L₄ and a capacitor 735 with capacitance C₃, forming a second secondary resonant circuit with a resonant frequency f₄. The second secondary circuit 738 also features an electrical actuator 736, comprising an elastomer 737 mechanically linked to a switch S₂. This switch S₂ is connected in series or parallel with the inductor 734 and the capacitor 735, regulating electrical flow in the second secondary circuit 738. When the switch S₂ is open, no electricity flows, leaving the second secondary circuit 738 unactuated. When the switch S₂ is closed, electricity flows, activating the second secondary circuit 738. Additional electrical actuators can be added to the circuit 700.

The first or second primary circuit 706, 736 detect the actuation state of the second secondary circuits 708, 738. This is monitored by the monitor circuit 714, which communicates the state to the control circuit 720 within a sealed medical device housing. The control circuit 720 links the activation of the electrical actuators 726, 736 to specific device functions. User activation is sensed across the sealed barrier, allowing for sterilization and reuse of the internal components while enabling disposal of the actuators 726, 736 and housing.

With reference to FIGS. 1-7, The monitor circuits 114, 314, 414, 514, 614, 714 discussed above may be implemented in a variety of configurations. These include voltage monitoring circuits, frequency detection circuits, or phase detection circuits. The outputs of the voltage monitor, frequency detector, or phase detector circuits can be provided to the control circuit 120, 320, 420, 520, 620, 720 to detect the switch S closure.

In various embodiments, the monitor circuit 114, 314, 414, 514, 614, 714 can include, without limitation, a variety of voltage monitor circuits. One example of a voltage monitor circuit is a Zener diode-based voltage monitor. These are simple voltage monitoring circuits that can be built using a Zener diode. The Zener diode is connected in reverse bias across the voltage to be monitored. When the input voltage exceeds the Zener voltage, the diode conducts, and this can be used to trigger a response, such as triggering a transistor coupled to the control circuit 120, 320, 420, 520, 620, 720.

Another example of a voltage monitor circuit is a comparator-based voltage monitor circuit. A comparator compares the monitored voltage to a reference voltage. If the monitored voltage exceeds (or drops below) the reference, the comparator output changes state, which can be used to trigger the control circuit 120, 320, 420, 520, 620, 720 or switch states in a control system to detect the switch S closure. Another example circuit could use an operational amplifier (op-amp) in a comparator configuration.

Another example of a voltage monitor circuit is a window comparator circuit voltage monitor circuit. A window comparator circuit uses two comparators to monitor if the voltage stays within a specified range (between two thresholds). If the voltage goes outside this range, the output of the comparators will trigger the control circuit 120, 320, 420, 520, 620, 720 to detect the switch S closure.

Another example of a voltage monitor circuit is a microcontroller-based or microprocessor-based voltage monitor circuit. A microcontroller or microprocessor with an integrated analog-to-digital converter (ADC), or coupled to an ADC, can be programmed to monitor the voltage continuously. The ADC measures the voltage, and the microcontroller's/microprocessor's software compares it to predefined limits, triggering actions if the voltage is out of range. The signal can be provided to the control circuit 120, 320, 420, 520, 620, 720 to detect the switch S closure.

Other examples of voltage monitor circuits are dedicated voltage monitoring integrated circuits including voltage reference circuits that can be used in conjunction with comparators or microcontrollers/microprocessors to monitor voltage levels. Such circuits provide a stable reference voltage for accurate monitoring. Some dedicated voltage monitoring integrated circuits include overvoltage and undervoltage monitors with dual-channel monitors with adjustable thresholds for overvoltage and undervoltage detection. Such monitor circuits can monitor two independent power sources and trigger a response to the control circuit 120, 320, 420, 520 if either channel exceeds its set limits to detect the switch S closure.

Yet another example of a voltage monitor circuit is a voltage divider coupled with a comparator. This circuit uses a voltage divider to scale down the monitored voltage to a level that can be compared with a reference voltage using a comparator. The comparator output will indicate if the voltage is above or below the desired level. The output can be provided to the control circuit 120, 320, 420, 520, 620, 720 to detect the switch S closure.

Another example of a voltage monitor circuit is a Schmitt trigger voltage monitor circuit. A Schmitt trigger circuit, which is a comparator with hysteresis, can be used to monitor voltage levels with noise immunity. The circuit changes its output only when the input crosses the upper or lower thresholds, which prevents spurious switching due to small voltage fluctuations. The output can be provided to the control circuit 120, 320, 420, 520, 620, 720 to detect the switch S closure.

Another example of a voltage monitor circuit is a relay-based voltage monitor circuit. This circuit uses a relay along with a Zener diode or comparator to monitor voltage levels. When the voltage exceeds or drops below the preset limit, the relay is triggered to trigger the control circuit 120, 320, 420, 520, 620, 720 to detect the switch S closure.

In other embodiments, the monitor circuit 114, 314, 414, 514, 614, 714 can include a variety of frequency detector circuits. One example of a frequency detector circuit is a zero-crossing detector circuit. A zero-crossing detector circuit is a type of frequency detector that identifies the points where a waveform crosses the zero-voltage level. The time interval between consecutive zero-crossings is inversely proportional to the signal frequency. The output can be provided to the control circuit 120, 320, 420, 520, 620, 720 to detect the switch S closure.

Another example of a frequency detector circuit is a frequency-to-voltage converter circuit. This circuit converts the frequency of an input signal into a corresponding DC voltage level. The output voltage is proportional to the input frequency. This signal can be provided to the control circuit 120, 320, 420, 520, 620, 720 to detect the switch S closure.

Another example of a frequency detector circuit is a phase-locked loop (PLL) with a frequency comparator circuit. A PLL can be used as a frequency detector by comparing the frequency of the input signal with a reference frequency and adjusting the output frequency until they both match. The output can be provided to the control circuit 120, 320, 420, 520, 620, 720 to detect the switch S closure.

Another example of a frequency detector circuit is a digital frequency counter circuit. A digital frequency counter circuit uses a microcontroller/microprocessor or a digital circuit to count the number of cycles of a periodic signal over a fixed time period, effectively measuring its frequency. The output of the voltage monitor circuit can be provided to the control circuit 120, 320, 420, 520, 620, 720 to detect the switch S closure.

In other embodiments, the monitor circuit 114, 314, 414, 514, 614, 714 can include a variety of phase detector circuits. One example of a phase monitor circuit is a phase detector circuit such as an exclusive OR (XOR) gate phase detector circuit. An XOR gate can act as a phase detector by taking two digital signals and outputting a signal that is high when the inputs differ. The average output voltage is proportional to the phase difference between the two inputs. The output of the voltage monitor circuit can be provided to the control circuit 120, 320, 420, 520, 620, 720 to detect the switch S closure.

Another example of a phase detector circuit is a multiplier-based phase detector. This analog circuit multiplies two signals together. The output is a sum and difference frequency component, with the difference frequency corresponding to the phase difference between the two signals. An ADC can be used to convert the analog signal and provide it to the output of the voltage monitor circuit can be provided to the control circuit 120, 320, 420, 520, 620, 720 to detect the switch S closure.

Another example of a phase detector circuit is phase-frequency detector (PFD). A PFD detects both phase and frequency differences between two signals. It outputs pulses whose width corresponds to the phase difference, and the sign of the pulse indicates whether one signal is leading or lagging. These pulses can be input into the control circuit 120, 320, 420, 520, 620, 720 to detect the switch S closure.

Another example of a phase detector circuit is an edge-triggered phase detector circuit. This detector uses flip-flops to capture the rising or falling edges of two signals. The relative timing of these edges indicates the phase difference. The output of the voltage monitor circuit can be provided to the control circuit 120, 320, 420, 520, 620, 720 to detect the switch S closure.

Each of these monitor circuits 114, 314, 414, 514, 614, 714 can be tailored to specific applications depending on the required accuracy, complexity, and response time. The choice of circuit will depend on the specific needs of the application, such as whether the focus is on simplicity, cost, or precision.

FIG. 8 depicts an electronic medical device 850, in accordance with at least one aspect of the present disclosure. The device 850 is divided into disposable and reusable components to enhance convenience and reduce waste. The reusable parts are designed to endure sterilization and autoclaving.

The disposable electronic medical device 850 features a housing 824 and handle 825 that are disposable. The electronic components, including the circuit 800, are reusable and sealed within a durable housing 822 that withstands sterilization and autoclaving. This design reduces waste and enhances cost-effectiveness. The electronic housing 822 can be removed from the device's housing 824.

The electronic circuit 800 receives input from a user-operated electrical actuator 826 positioned on the exterior wall of the housing 824. This input controls the motors 852 and other electronic functions. Wired connections are impractical because the housing 822, which contains the electronic circuit 800, must be sealed to withstand sterilization and autoclaving. Typically, the device rotates a drive shaft 858 within an outer tubing 854 to operate an end effector 856.

Various embodiments of the configuration and operation of the circuit 800 is described above in FIGS. 1-7. In summary, the circuit 800 includes an inductor 802 that is contained within an inner wall of the sealed housing 822. The inductor 802 is positioned at a distance (d1) that is proximate to an inductor 804 positioned on an exterior wall of the medical device housing 824. The inductor 804 is a component of an external electrical actuator 826 that is affixed to the exterior wall of the medical device housing 824 using an adhesive or other suitable techniques. The electrical actuator 826 also includes an elastomer 827 mechanically linked to a switch S₁. This switch S₁ is connected in series or parallel with the inductor 804 and optionally a capacitor, to regulate electrical flow in the secondary circuit 808. When the switch S₁ is open, no electricity flows, leaving the first secondary circuit 808 unactuated. When the switch S₁ is closed, electricity flows, activating the first secondary circuit 808. Additional electrical actuators can be added to the circuit as needed. The circuit 800 detects the closure of the switch S₁ when a mutual inductance M is established between the inductors 802, 804. Once the closure of the switch S₁ is detected by a monitor circuit portion of the circuit 800, which communicates the switch S₁ closure to a control circuit portion of the circuit 800 to control the operation of the motor 852, for example.

The description of the circuit 800 in FIGS. 1-7 outlines its configuration and operation. The circuit 800 includes an inductor 802 within the inner wall of the sealed housing 822, positioned proximate to an inductor 804 attached to the exterior wall of the medical device housing 824. This inductor 804 is part of an external electrical actuator 826, attached using adhesive or other methods. The actuator 826 features an elastomer 827 mechanically linked to a switch S₁, which may be connected in series or parallel with the inductor 804 and optionally a capacitor, to control electrical flow in the secondary circuit 808. When switch S₁ is open, the circuit 808 remains unactuated; when closed, it activates. Additional actuators can be added as needed. The circuit 800 detects the closure of switch S₁ through mutual inductance M between the two inductors 802, 804. This closure is communicated by a monitor circuit within the circuit 800 to a control circuit, which can then operate the motor 852, for example.

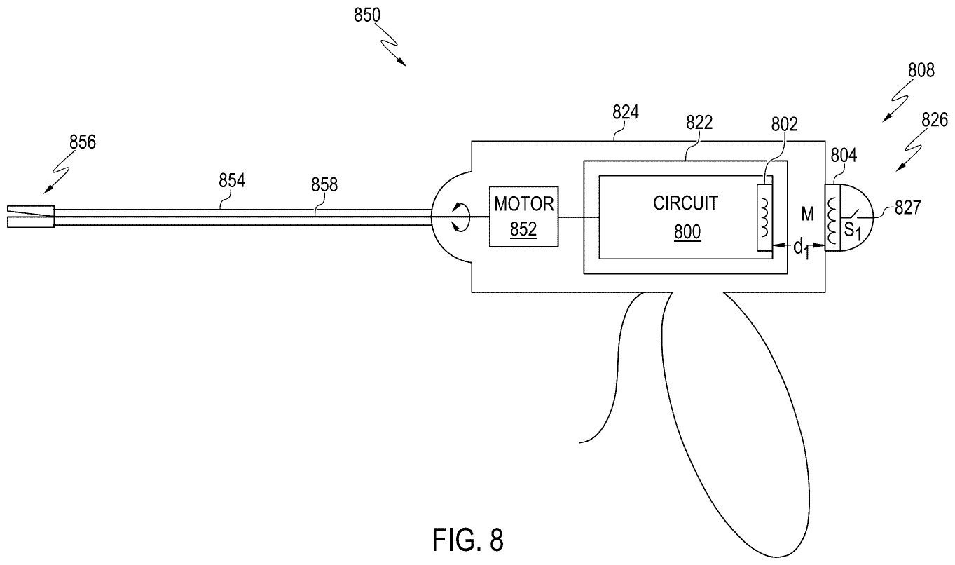

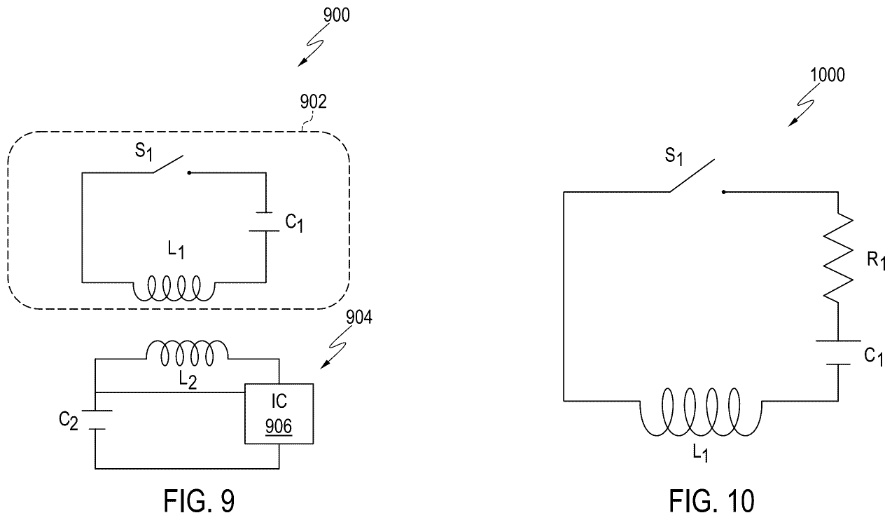

FIG. 9 depicts a circuit 900 including a primary circuit 902 and a secondary circuit 904, in accordance with at least one aspect of the present disclosure. The primary circuit 902 includes a wireless button that can be attached to or mounted on the exterior medical device housing, such as the medical device 850 shown in FIG. 8. The secondary circuit 904 is located near the primary circuit 902 but is housed within the internal space defined by the medical device housing.

The primary circuit 902 features a switch S₁ connected in series with a first LC resonant circuit, which includes a first inductor L₁ and a first capacitor C₁. The secondary circuit 904 contains a second LC resonant circuit with a second inductor L₂ and a second capacitor C₂. The first and second inductors L₁, L₂ are arranged to enable mutual inductance when the switch S₁ is closed, allowing current to flow through the primary circuit 902. This mutual inductance is detected by the control circuit 906, which then activates a function of the medical device associated with the closure of the switch S₁.

FIG. 10 depicts a primary circuit 1000, in accordance with at least one aspect of the present disclosure. The primary circuit 1000 can be attached to or mounted on the exterior of a medical device, similar to the medical device 850 in FIG. 8. The primary circuit 1000 includes a switch S₁ connected in series with an LCR resonant circuit, which includes a first inductor L₁, a first capacitor C₁, and a first resistor R₁. When the switch S₁ is closed, current flows through the circuit, and the first inductor L₁ couples with a second inductor, such as L₂ in FIG. 9, via mutual inductance.

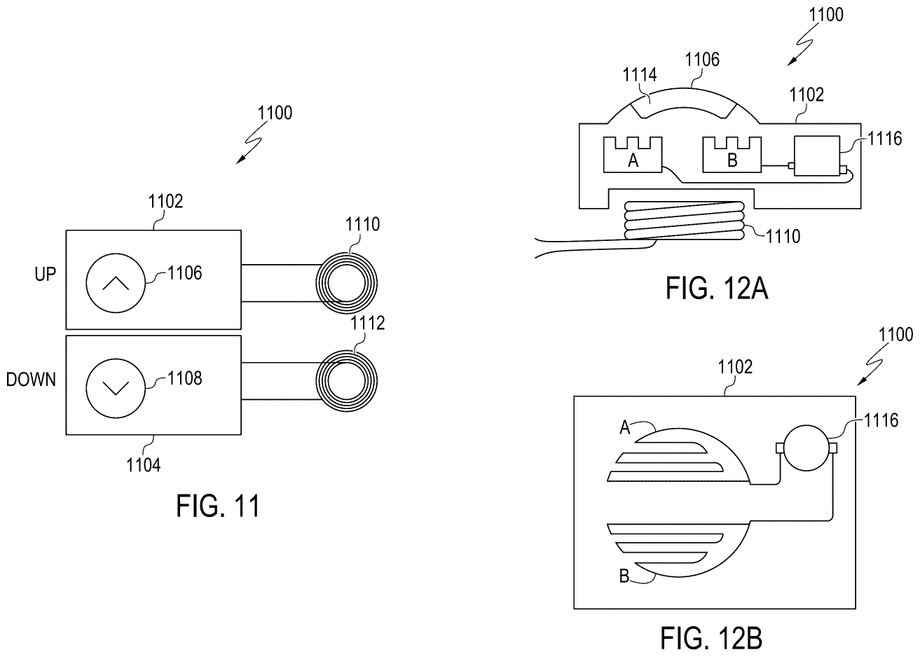

FIG. 11 shows a pair of external buttons 1100 designed for a medical device, in accordance with at least one aspect of the present disclosure. This pair of external buttons 1100 include a first button 1102 and a second button 1104, which provide UP/DOWN articulation control for a medical device, like the medical device 850 in FIG. 8. FIG. 12A presents a side elevation section view of the first button 1102, highlighting its internal features, while FIG. 12B offers a plan view of these features. Since the second button 1102 is similar to the first button 1104, additional views of the second button 1104 are not necessary.

Referring now to FIGS. 11 and 12A, B, the first button 1102 includes a first dome membrane switch 1106. The switch 1106 operates as an electrical switch utilizing a dome structure made from an elastomer such as rubber or silicone, or in some cases metal. The dome collapses when pressed, allowing the electrical conductor 1114 to establish make electrical contact with electrically conductive circuit traces A and B. These traces, which may be carbon contacts, form an electrical pathway when trace A is electrically connected to trace B, activating a first resonant circuit that includes a first inductor 1110, along with components such as capacitors or resistors as previously described. In this example, pressing the first dome membrane switch 1106 activates a specific function of the medical device, such as UP-articulation.

Similarly, the second button 1104 contains a second dome membrane switch 1108, which functions in the same manner as the first dome membrane switch 1106. It features a dome made from an elastomer like rubber or silicone, or in some instances metal. When the dome is pressed, it collapses, allowing an electrical conductor to contact electrically conductive circuit traces, creating a pathway that activates a second resonant circuit. This second inductor 1112 and additional components such as capacitors or resistors, as previously described. Pressing the second dome membrane switch 1108 activates another medical device function, such as DOWN-articulation.

Unless specifically stated otherwise as apparent from the foregoing disclosure, it is appreciated that, throughout the present disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

As used herein, the singular form of “a”, “an”, and “the” include the plural references unless the context clearly dictates otherwise.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material. None is admitted to be prior art.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

1-15. (canceled)

16. An electronic medical device, comprising:
a disposable housing;
a primary circuit disposed in a sealed housing removably connectable to the disposable housing, the primary circuit comprising a primary inductor with inductance L₁ to generate a magnetic field in response to applied electrical energy; and
a secondary circuit positioned on an exterior wall of the disposable housing and proximal to the primary circuit, the secondary circuit comprising:
an electrical actuator comprising a secondary inductor with inductance L₂ arranged in proximity to the primary inductor to induce electrical energy in the secondary inductor via mutual inductance M when the primary inductor is energized, wherein the electrical actuator is operable to control the electrical flow through the secondary inductor based on the induced electrical energy;
wherein magnetic coupling between the primary inductor and the secondary inductor is defined by the mutual inductance M, and wherein the electrical actuator is configured to control energy transfer from the primary inductor to the secondary inductor to control the induced electrical energy in the secondary circuit.

17. The electronic medical device of claim 16, wherein the primary circuit comprises a direct current (DC) voltage source to energize the primary inductor.

18. The electronic medical device of claim 16, wherein the primary circuit comprises an alternating current (AC) voltage source to energize the primary inductor.

19. The electronic medical device of claim 16, wherein the electrical actuator comprises a switch connected in series or in parallel with the secondary inductor.

20. The electronic medical device of claim 19, wherein the electrical actuator comprises an elastomer mechanically linked to the switch.

21. The electronic medical device of claim 20, wherein the electrical actuator is affixed to the exterior wall of the disposable housing with an adhesive.

22. An electronic medical device, comprising:
a disposable housing;
a primary circuit disposed in a sealed housing removably connectable to the disposable housing, the primary circuit comprising:
a primary inductor with inductance L₁ to generate a magnetic field in response to applied electrical energy; and
a monitor circuit configured to detect changes in electrical characteristics of the primary inductor;
a secondary circuit positioned on an external wall of the disposable housing and proximal to the primary circuit, the secondary circuit comprising:
an electrical actuator comprising a secondary inductor with inductance L₂ positioned in proximity to the primary inductor to induce mutual inductance M between the primary inductor and the secondary inductor, wherein the electrical actuator is operable to control electrical flow through the secondary inductor based on induced voltage;
wherein the changes in the electrical characteristics of the primary inductor are based on an activation state of the electrical actuator.

23. The electronic medical device of claim 22, wherein the monitor circuit is configured to detect the changes in electrical characteristics of the primary circuit, including changes in voltage, current, impedance, frequency, or phase, or combinations thereof, based on whether the electrical actuator is open or closed.

24. The electronic medical device of claim 23, wherein the primary circuit comprises a direct current (DC) source to energize the primary inductor or an alternating current (AC) source to energize the primary inductor.

25. The electronic medical device of claim 23, wherein the electrical actuator comprises a switch and a frequency tuning element.

26. The electronic medical device of claim 25, wherein the switch is connected in series or in parallel with the frequency tuning element.

27. An electronic medical device, comprising:
a disposable housing;
a primary circuit disposed in a sealed housing removably connectable to the disposable housing, the primary circuit comprising:
a first inductor L₁ and a first capacitor C₁ to form a primary resonant circuit with a resonant frequency f₁;
a monitor circuit operatively connected to the primary resonant circuit and configured to measure an electrical characteristic of the primary resonant circuit;
a secondary circuit positioned on an exterior wall of the disposable housing arranged in proximity to the primary circuit to enable magnetic coupling through mutual inductance M between the primary and secondary circuits, the secondary circuit comprising:
an electrical actuator comprising a second inductor L₂ and a second capacitor C₂ to form a second resonant circuit with a resonant frequency f₂, wherein the electrical actuator is configured to activate the secondary circuit;
wherein the primary circuit and the secondary circuit are coupled by mutual inductance M based on a state of the electrical actuator; and
wherein the monitor circuit is configured to detect a response of the primary circuit based on the state of the electrical actuator.

28. The electronic medical device of claim 27, comprising a frequency-swept alternating current (AC) signal source to generate a signal with a frequency that varies over a predetermined range, including the resonant frequency f₁.

29. The electronic medical device of claim 28, wherein the monitor circuit is configured to detect the electrical characteristics of the primary circuit over the frequency range of the signal.

30. The electronic medical device of claim 28, wherein the primary resonant circuit exhibits a first response when the electrical actuator is open and a second response when it is closed, and wherein the monitor circuit is configured to detect changes between the first and second responses.

31. The electronic medical device of claim 30, wherein the monitor circuit is configured to detect a state of the electrical actuator based on the changes between the first and second responses during the frequency sweep.

32. The electronic medical device of claim 27, comprising an additional secondary circuit positioned on an exterior wall of the disposable housing arranged in proximity to the primary circuit to enable magnetic coupling through mutual inductance M between the primary and the additional secondary circuits, the additional secondary circuit comprising:
a second electrical actuator comprising a third inductor L₃ and a third capacitor C₃ to form a third resonant circuit with a resonant frequency f₃, wherein the second electrical actuator is configured to activate the additional secondary circuit;
wherein the primary circuit and the additional secondary circuit are coupled by mutual inductance M based on the state of the second electrical actuator; and
wherein the monitor circuit is configured to detect a response of the primary circuit based on the state of the second electrical actuator.

33. The electronic medical device of claim 32, wherein the primary resonant circuit exhibits a first response when the second electrical actuator is open and a second response when it is closed, and wherein the monitor circuit is configured to detect changes between the first and second responses.

34. The electronic medical device of claim 33, wherein the monitor circuit is configured to detect a state of the second electrical actuator based on the changes between the first and second responses during the frequency sweep.

35. The electronic medical device of claim 27, comprising:
a second primary circuit, comprising:
a third inductor L₃ and a third capacitor C₃ to form a third resonant circuit with a resonant frequency f₃;
a second monitor circuit operatively connected to the third resonant circuit and configured to measure an electrical characteristic of the third resonant circuit;
a second secondary circuit positioned on an exterior wall of the disposable housing arranged in proximity to the second primary circuit to enable magnetic coupling through mutual inductance M between the second primary and second secondary circuits, the second secondary circuits comprising:
a second electrical actuator comprising a fourth inductor L₄ and a fourth capacitor C₄ to form a fourth resonant circuit with a resonant frequency f₄, wherein the second electrical actuator is configured to activate the second secondary circuit;
wherein the second primary circuit and the second secondary circuit are coupled by mutual inductance M based on the state of second electrical actuator;
wherein the second monitor circuit is configured to detect a response of the second primary circuit based on the state of the second electrical actuator.
